(19) United States
(12) Patent Application Publication
(10) Pub. No.: US 2026/0183536 A1
(43) Pub. Date: Jul. 2, 2026

(54) WEARABLE NEUROSTIMULATION GARMENT FOR POSITIONING ELECTRONICS UNIT

(71) Applicant: Noctrix Health, Inc., Pleasanton, CA (US)

(72) Inventors: John Craig Colborn, Magnolia, TX (US); Jeffrey John Schirm, Monroeville, PA (US)

(21) Appl. No.: 19/430,463

(22) Filed: Dec. 23, 2025

Related U.S. Application Data
(60) Provisional application No. 63/740,689, filed on Dec. 31, 2024.

(51) Int. Cl. A61N 1/04; A61N 1/36

(52) U.S. Cl. CPC A61N 1/0484; A61N 1/0456; A61N 1/36003; A61N 1/36034

(57) ABSTRACT

A garment for holding a neurostimulation device can include a thermal insulation layer with textile configured to reduce thermal transfer between the device and patient skin. The garment can include a cavity, sized to partially encase the neurostimulation device, and an electrode aperture arranged to expose device electrodes to an external target location. An affixation feature can pair with a corresponding feature of the neurostimulation device such as to align and attach the device at a specified location within the cavity. The cavity and affixation feature can maintain device attachment and alignment when subjected to specified shear forces or bending deflections.

### PRIORITY

This application claims the benefit of priority to U.S. Provisional Patent Application Ser. No. 63/740,689, filed Dec. 31, 2024, which is incorporated by reference herein in its entirety.

### BACKGROUND

Electrical nerve stimulation can be used to treat one or more conditions, such as chronic or acute pain, epilepsy, depression, bladder disorders, or inflammatory disorders. Certain neurological disorders can be attributed to overactivity of sensory or other peripheral nerve fibers which can disrupt quality of life, or the processing of such neural activity in the brain. Restless Legs Syndrome (RLS) and Periodic Limb/Leg Movement Disorder (PLMD) are two such neurological conditions that can significantly affect sleep in human subjects. RLS (which can also be called Willis-Ekbom Disease (WED)) subjects can experience uncomfortable tingling sensations in their lower limbs (legs) and, less frequently in the upper limbs (arms). RLS is characterized by an uncontrollable urge to move the affected limb(s). Such sensations can often be temporarily relieved by moving the limb voluntarily but doing so can interfere with the RLS subject's ability to fall asleep. PLMD subjects can experience spontaneous movements of the lower legs during periods of sleep, which can cause the PLMD subject to wake up. RLS can be a debilitating sleep disorder and can be comorbid with other sleep disorders such as insomnia or sleep apnea syndrome (SAS).

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 depicts an example of a limb wearable neurostimulation device for treating at least one of RLS or PLMD.

FIG. 2A depicts an electrostimulation electronics unit for use as part of a neurostimulation device.

FIG. 2B depicts insertion of an electrostimulation electronics unit into an opening of a removable strap.

FIG. 2C depicts an example of a limb wearable neurostimulation device for treating at least one of RLS or PLMD.

FIG. 3 is an exploded view of an example of a limb wearable neurostimulation device for treating at least one of RLS or PLMD.

FIG. 4A depicts an electrostimulation electronics unit from an end user point of view for insertion into an opening of a removable strap.

FIG. 4B depicts an assembled electrostimulation device, including an electrostimulation electronics unit having been user-inserted into a removable strap, from an end user point of view.

FIG. 5 depicts an example of a display of an electrostimulation device.

FIG. 6A depicts an example of an affixation feature of a removable strap, sized and shaped to pair with a corresponding aperture of an electrostimulation electronics unit.

FIG. 6B depicts an example of an affixation feature of a removable strap, sized and shaped to pair with a corresponding aperture of an electrostimulation electronics unit.

### DETAILED DESCRIPTION

Neurostimulation therapy can provide a treatment modality for disorders such as Restless Legs Syndrome (RLS) and Periodic Limb Movement Disorder (PLMD). For example, electrical stimulation can be delivered through electrodes placed on a patient's skin can activate underlying neural pathways and muscle tissue. Electrostimulation can employ an electronic device that can generate and deliver electrical waveforms with specified characteristics, such as at controlled current, frequency, and amplitude parameters. Such an electrostimulation device can include an onboard battery and be end user wearable, such as on a limb. Successful therapeutic delivery of neurostimulation can involve positioning and maintaining electrodes at precise locations on a patient's limb, which can be challenging using certain wearable devices that involve end-user configuration.

In an example, a wearable neurostimulation device is contained in an elongate band or strap, such that the electronic device for generating the waveform is integrated within the wearable garment. A challenge with such a wearable device having integrated electronics is that the garment portion cannot be easily washed or replaced, such that prolonged use of the wearable neurostimulation device can soil or otherwise wear on the garment portion and replacement of the garment portion generally involves replacement of the entire device. This can be wasteful, as the electronics integrated within the wearable neurostimulation device can still function as desired, despite the garment portion needing replacement. On the other hand, certain approaches to separating the electronic device from the garment portion can involve significant challenges in assembly of the two to form a functioning, wearable neurostimulation device, as the capability to properly assemble, disassemble, and align device features by the end user can be limited. Further, certain approaches involving electrical connections between such a garment portion, an electronics device, and electrodes can require end user connection and disconnection of cords or plugs, which can be burdensome and sometimes even involve a possibility for end user error which can damage the electronic device.

This document describes a wearable neurostimulation system including a garment portion for securing a user-removable neurostimulation device against patient skin, within requiring electrical connection between the garment and the neurostimulation device. In an example, the garment portion can include a thermal insulation layer comprising a textile configured to reduce thermal transfer between the neurostimulation device and patient skin. For example, the thermal insulation layer may comprise various foam materials, including polyethylene foam, polyurethane foam, polystyrene foam, or ethylene-vinyl acetate foam. The garment portion can include a pocket defining a cavity specifically sized and shaped to partially encase the neurostimulation device. An electrode aperture in the pocket enables exposure of device electrodes to the target location on the patient's skin.

In an example, the garment can include or use an affixation feature designed to pair with a corresponding feature on the neurostimulation device, such as to facilitate precise alignment and attachment at a specified location within the cavity. For an example, the affixation feature includes an affixation aperture sized to receive an anchor projection from the neurostimulation device. Such features can facilitate maintaining a desired device positioning and alignment, even when subjected to specified shear forces ranging from 0.4 to 45 newtons or bending deflections within 3.5-inch diameter to 9-inch diameter ranges. Herein, the bending deflection can be described as radial deflection, as if the neurostimulation device is being “rolled” toward a tubular shape, with the diameter of the “tube” being within the recited diameter range. In an example, the removable strap 218 can provide a viewing window 603 that reveals a display 605 on the device's anchor projection, e.g., allowing an end user to view certain specified limb settings such as a chirality setting of the neurostimulation device. Such a viewing window 603 can be medially arranged on the wearable garment such as to maintain visibility during bending of the garment.

The neurostimulation device can include a flexible, elongate housing designed for user insertion into the garment cavity. The device can include (e.g., housed within the elongate housing) a printed circuit board (PCB) with a neurostimulation electrical waveform generator capable of producing tonic motor activation (TOMAC) excitation waveforms. Such TOMAC excitations waveforms can involve constant current with frequencies between 350 Hz and 10,000 Hz and zero-to-peak current amplitudes of 10 to 80 milliamperes. In an example, the elongate housing can be formed at a thickness and of a material such as to provide sufficient rigidity for easy insertion while resisting buckling, and the device can deliver therapy without requiring connection to external devices. In an example, the PCB and an electronics battery can be internally positioned different locations along a length of the housing, such as to provide articulation or bending of the elongate housing between the PCB and the electronics battery.

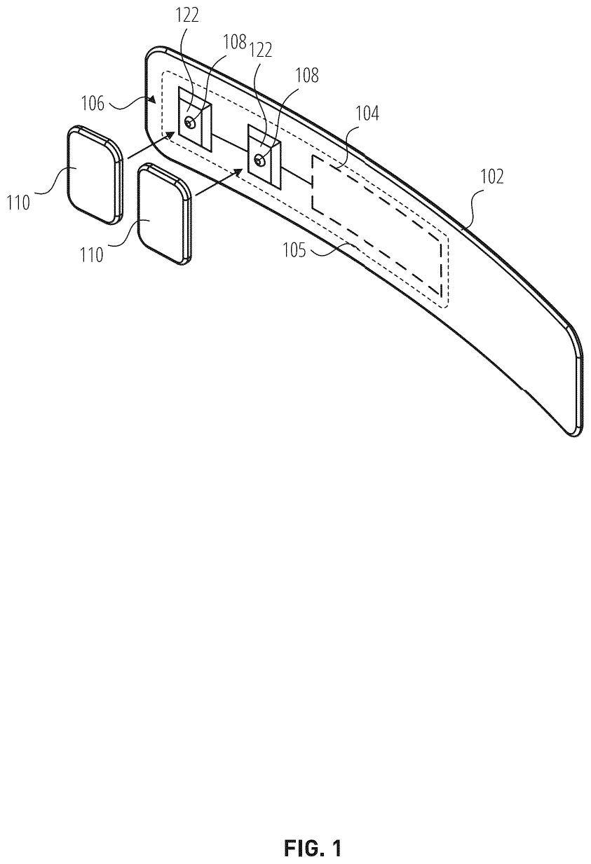

FIG. 1 depicts an example of a limb wearable neurostimulation device for treating at least one of RLS or PLMD. In an example, an electrostimulation therapy system can include a wearable electrostimulation device 102 including, e.g., an electrostimulation electronics unit 104, and one or more electrostimulation electrodes 106. For example, the wearable electrostimulation therapy device 102 can deliver electrostimulation therapy to skin of a subject via charge-dispersing materials such as the electrode pads 110, which help form the electrodes 106. In an example, the electrode pads 110 can be removably couplable to the wearable electrostimulation device 102. The electrode pads 110 can each be attached to a pairing surface 122 of the wearable electrostimulation device 102. The pairing surface 122 can include the electrode terminal 108 e.g., disposed therein, and the electrode terminal 108 can be electrically connected to the electrostimulation electronics unit 104. In an example, two or more electrode pads 110 are each paired to corresponding pairing surfaces 122 including one electrode terminals 108. In another example, one electrode pad 110 can be paired to an electrode pairing surface 122 containing more than one electrode terminal 108, or one electrode pad can span multiple electrode pairing surfaces 122 containing one or more electrode terminals 108. In an example, the electrode pad 110 can be removable for, e.g., hygienic maintenance, electrode maintenance such as rehydrating, or disposal.

The wearable electrostimulation device 102 can be worn by the subject and can include or use the electrostimulation electronics unit 104 coupled to the electrodes 106, such as for transcutaneously delivering an electrostimulation signal. The wearable electrostimulation device 102 can be sized and shaped to be able to be attached or held to a body location of the subject, e.g., a leg, arm, foot, waist, neck, head, or chest of the subject. In an example, the wearable electrostimulation device 102 can include or use a strap to help hold the electrodes 106 to the skin of the subject. While electrodes are generally described herein with a focus on providing electrostimulation to a subject, the electrodes can alternatively or additionally be used such as to help detect or measure one or more biosignals or biopotentials from the subject. A particular electrode 106 can include or use an electrode terminal 108 and an electrode pad 110. For example, the electrode terminal 108 can receive a capacitively-coupled (e.g., coupled using series DC-blocking capacitors, e.g., in a charge-balanced arrangement) electrostimulation signal from the electrostimulation electronics unit 104, and can deliver a resulting electrostimulation signal to the skin of the subject, such as via the electrode pad 110. In an example, multiple electrodes, such as two electrodes 106, can be used. For a bipolar electrode example having two electrodes 106, this can a first electrode that can serve as an anode and a second electrode, such as which can serve as a cathode. Also, a plurality of electrodes 106 can be arranged to form a multi-electrode group, matrix, or array such as for one or both of sensing or for delivering the electrostimulation signal to the skin of the subject. In an example, each electrode terminal 108 can be an electrode contact fixed to the wearable electrostimulation device 102 and each corresponding electrode pad 110 can be removably couplable to the device 102. In other examples, the electrode pad can be fixed to the wearable electrostimulation device 102. Because the current density of the electrostimulation signal at the electrode terminal 108 may be larger than desired, the electrode pad 110 can include an embedded or other arrangement of electrical conductors that can help distribute the electrostimulation signal current over a larger effective surface area for delivery to the subject at the skin-electrode interface. The electrode pad can be formed of a hydrogel, a hydrophilic polymer such as polyvinyl alcohol (PVA), carbon, textiles, or other types of conductive or dielectric gels, a polymer, or a textile. In an example, two wearable electrostimulation devices 102 can be worn bilaterally, such as on different limbs of a patient to provide bilateral electrostimulation.

An individual wearable electrostimulation device 102 can include an electrostimulation electronics unit 104 communicatively coupled to the electrostimulation electrode(s) 106. In an example, one or both of the electrostimulation electronics unit 104 and the electrostimulation electrodes 106 can be included in or at least partially housed within a removeable neurostimulation device 105, and the device 105 can be end user removable from the rest of the device 102, such as removable from the strap. The wearable electrostimulation device 102 can be worn by the subject and can include or use the electrostimulation electronics unit coupled to the electrodes, such as for transcutaneously delivering an electrostimulation signal. Herein, the term high frequency electrostimulation (HFS) can refer to certain high-frequency (greater than 400 Hertz, such as greater than 1 kilohertz) electrostimulation waveforms capable of inducing tonic motor activation (TOMAC) when applied at an external target body location of the patient. This is distinct from other approaches to electrostimulation, such as TENS or implantable devices which operate at considerably different parameters which are generally not compatible with sleep or for treating RLS or PLMD, such as parameters that involve sensory perception of the patient and that do not induce TOMAC in a patient muscle. In an example, the HFS devices and methods herein include use of a capacitive coupling to apply HFS which induces TOMAC in a particular muscle without attempting to surround current injected in skin with a large opposite current as in TENS paradigms in, e.g., evident during electromyogram monitoring.

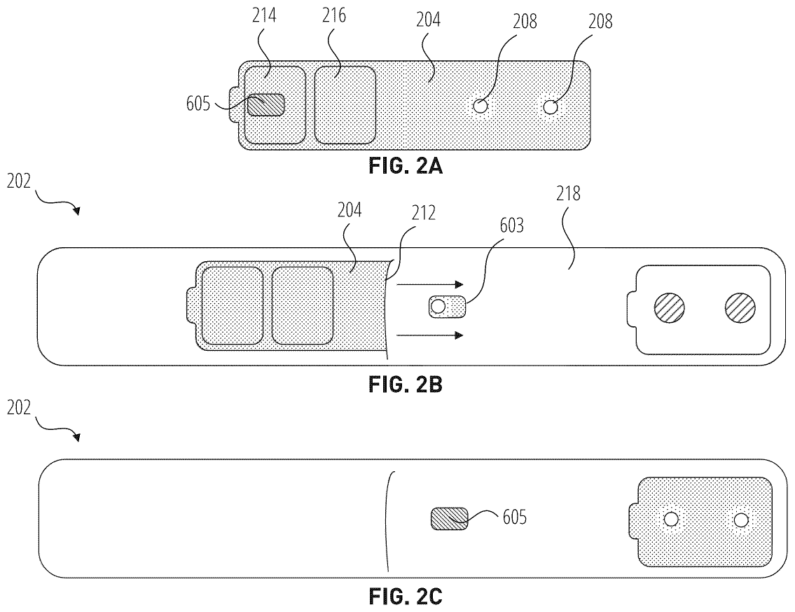

FIG. 2A depicts an electrostimulation electronics unit for use as part of a neurostimulation device. The electrostimulation electronics unit 204 can be similar in many respects to the electrostimulation electronics unit 104 of FIG. 1. The components, structures, configuration, functions, etc. of unit 204 can therefore be the same as or substantially similar to that described in detail above with reference to unit 104. In an example, the electrostimulation electronics unit 204 can including a main printed circuit board (PCB) housing 214 and a battery housing 216. In an example, the PCB housing 214 and the battery housing 216 can be located adjacent to each other, such as encased in a housing top 220 and housing bottom 224 of a casing or chassis of the electrostimulation electronics unit 204.

The electrostimulation electronics unit can include waveform generation circuitry configured to supply an alternating current (AC) electrostimulation signal for delivery to the skin at an electrode-skin interface. The electrostimulation waveform can be supplied at a frequency between about 350 hertz (Hz) to about 10,0000 Hz such as for treating Restless Leg Syndrome (RLS) or Periodic Limb Movement Disorder (PLMD), such as described in Charlesworth U.S. Pat. No. 11,103,591 and also as described in Raghunathan WIPO application number PCT/US2024/024116, each of which are hereby incorporated by reference in their entirety. The electrostimulation electronics unit 204 can generate TOMAC excitation waveform for delivery, via the one or more electrodes, to the external target body location. Herein, the term “TOMAC excitation waveform” means a waveform having the parameters to induce tonic motor activation (e.g., activating proprioceptive afferents) in a patient muscle when applied at an exterior location of a patient (e.g., the outermost layer of patient skin). In an example, waveform generation circuitry can generate a TOMAC waveform having a frequency between 350 Hertz (Hz) and 10,000 Hz and at a specified first current between 5 milliamperes (mA) and 80 mA. For example, the TOMAC waveform can be generated at a frequency within a range of about 1 kHz (kilohertz) and 5 kHz, or about 2 kHz. The TOMAC waveform can be generated via a power source supplying less than about 100 volts zero-to-peak voltage amplitude per phase, such as less than about 60 volts zero-to-peak voltage amplitude per phase. In an example, the TOMAC waveform can be generated having a duty cycle greater than about 25%, such as a duty cycle greater than about 40%, greater than about 50%, greater than about 75%, or greater than about 90%. In an example, the TOMAC waveform can deliver a specified root means squared (RMS) current (e.g., over a specified duration such as about 10 minutes (min), about 20 min, about 30 min, about 40 min, etc.). For example, the RMS current can be between about 10 mA and about 40 mA. This approach is distinct from that of other TENS devices, which generally exhibit an RMS current between about 3 mA and about 5 mA and involve much lower frequencies (e.g., within a range of about 1 Hz to about 300 Hz) and lower duty cycles (e.g., within a range of about 2% to about 15%). The present inventors have recognized the benefits of delivering such a high frequency, high duty cycle current, including delivery of sufficient charge to induce TOMAC in the patient without discomfort or injury to the patient. Such a physiological response is not able to be induced by other electrostimulation approaches, such as TENS.

FIG. 2B depicts insertion of an electrostimulation electronics unit into an opening of a removable strap. In an example, the electrostimulation electronics unit 204 can be attached to, embedded within, or sized and shaped for mating with a strap, sleeve, clamp, or band to help hold the electrodes to the skin of the subject, the strap/unit 204 forming limb wearable neurostimulation device 202. For example, as depicted in FIG. 2, the electrostimulation electronics unit can be configured to couple with a removable strap 218. Alternatively or additionally, the wearable neurostimulation device 202 can include or use an adhesive or can connect to other items wearable by the subject, e.g., hats, clothing, etc.

The wearable electrostimulation device 202 can be attached or held to (e.g., via the removable strap 218) a body location of the subject, e.g., a leg, arm, foot, waist, neck, head, or chest of the subject at an external body location corresponding with a nerve target (e.g., at or near a peroneal nerve, at or near a sural nerve, etc.) of the subject skin for transcutaneous electrostimulation thereof. In an example, the electrostimulation electronics unit 204 can be insertable into a pocket defining a cavity of the removable strap 218 (e.g., through an opening 212 of the removable strap 218), such as to facilitate end-user entry and removal of the unit 204 into the removable strap 218. In an example, the removable strap 218 can be user-washable, such as made of a material that is machine washable without deforming or damaging the neurostimulation device 202. For example, the neurostimulation device 202 can be removed from the cavity of the strap 218 during washing or sanitizing of the removable strap 218. The use of a removable strap 218 can promote longevity of the neurostimulation device 202, such that the removable strap 218 can be replaced at a greater frequency than the electrostimulation electronics unit 204 over the course of months or years of neurostimulation therapy.

In an example, the removable strap 218 can be an end user-replaceable garment, such as including a thermal insulation layer (e.g., a textile, foam or a combination thereof). The thermal insulation layer can be arranged along the removable strap to reduce thermal transfer between the electrostimulation electronics unit 204 and patient skin. The thermal insulation layer can include, e.g., a one or more foam materials such as polyethylene foam, polyurethane foam, polystyrene foam, or ethylene-vinyl acetate foam, or a combination thereof.

FIG. 2C depicts an example of a limb wearable neurostimulation device for treating at least one of RLS or PLMD. The neurostimulation device 202 can be similar in many respects to the wearable electrostimulation device 102 of FIG. 1. The components, structures, configuration, functions, etc. of the neurostimulation device 202 can therefore be the same as or substantially similar to that described in detail above with reference to the device 102. The neurostimulation device 202 as a whole can be a wearable solution, such as featuring an elongate strap (e.g., the removable strap 218) that can substantially encircle the limb. The strap can be resizable to fit a plurality of different limb sizes, such as adjustable via a hook-and-loop connection, a buckle, a clip, etc. The neurostimulation device 202 as a whole can be substantially stretchable or resilient in at least one dimension, so as to be adaptable to general or specific wearers per unit of time. For example, the device may include an elongate portion, and the neurostimulation device may be provided as a bendable or articulated zone along the elongate portion, such that the strap is substantially conformal to a limb contour (e.g., around a human calf portion of a leg). In an example, the strap can be sized and shaped and formed of a material for flexibility and comfort, such as including a battery housing and an electronics housing arranged at different locations along the strap length to facilitate articulation when worn. The strap can hold the electrodes (e.g., including or attached to the electrode terminals 208), which are electrically connected to the electrostimulation electronics unit 204, against the patient's skin.

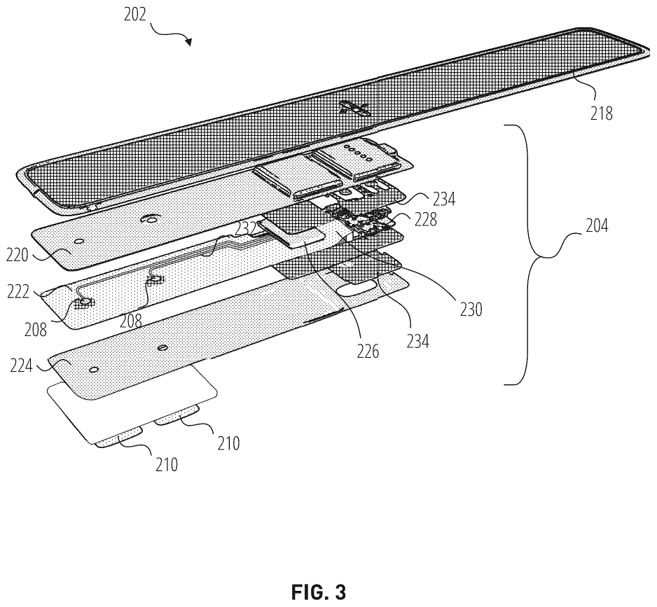

FIG. 3 is an exploded view of an example of a limb wearable neurostimulation device for treating at least one of RLS or PLMD. In an example, the neurostimulation device 202 can include or use a removable strap 218, electrode pads 210 (e.g., for electrical connection with respective electrode terminals 208), and an electrostimulation electronics unit 204 including a housing top 220, a housing bottom 224, main PCB 228, a flexible printed circuit board 222 (also herein called a flex circuit 222), and a battery 226. In an example, the neurostimulation device 202 can include one or more intermediate layers, such as one or more rubber contact layers 234 arranged facilitate transfer of end-user button pressing to, e.g., a contact on the main PCB 228.

Generating the above-described TOMAC excitation waveform that is compatible with sleep, particularly in a wearable, battery operated device, can be challenging in terms of heat dissipation, power consumption, efficiency, and therapeutic efficacy. For example, several electrostimulation and thermal regulation parameters (e.g., frequency, RMS current as a function of duty cycle, battery size, heat dispersion from to the patient from the neurostimulation device 202, total charge delivered to the patient, slew rate, can be described as “interdependent” parameters or mutually dependent variables. The neurostimulation device 202 involves optimizations which balance certain “trade-offs” of inherent factors in neurostimulation to arrive at an effective, safe, and portable (e.g., for use in at-home therapy) treatment for RLS or PLMD.

HFS, while providing a comfortable and efficacious waveform to patient skin, such benefits are provided at the “cost” of certain challenges. First, HFS involves significantly higher reactive power loss at an electrode-skin interface, due to a capacitance of human skin. For example, HFS can involve up to about 20× more reactive power loss than certain TENS waveforms, such as meeting a capacitive load within a range of about 5 nanofarads (nF) and about 150 nF, such as a skin tissue having a capacitance greater than about 100 nF). The present techniques to delivery of HFS, via the “TOMAC excitation waveform” involves (e.g., when compared to TENS and implantable neurostimulation) a relatively high amount of charge delivered per second (e.g., within a range of about 0.001 to about 0.04 coulombs per second) and at a relatively small charge per phase (about 1.25 E-7 to 6.3 E-6 coulombs per phase, e.g., within a range of about 0.1 to about 6.3 micro-coulombs per phase) such as depending on a duty cycle of the waveform. Such relatively high amounts of charge, alongside a relatively high RMS current (e.g., related to a duty cycle greater than about 25%) can overcome such the reactive power loss at the electrode-skin interface and thus overcome the capacitance of human skin despite the challenge of HFS.

To help promote the above benefits, the electrostimulation electronics unit 204 can include waveform generation circuitry to produce the TOMAC excitation waveform with a slew rate of less than about 25 microseconds (μs) rise-time and, alternatively or additionally, less than about 25 μs fall-time. Here, the slew rate of less than about 25 μs fall-time can be considerably faster than a typical capacitive discharge of a TENS device, which directly results in more total energy dispersed per phase. This, and optionally controlling a duty cycle of the TOMAC excitation waveform to be relatively high (e.g., a duty cycle greater than about 25%, such as a duty cycle greater than about 40%, greater than about 50%, greater than about 75%, or greater than about 90%) can promote a greater amount of charge delivered to the patient skin when compared to other approaches, such as to help overcome certain challenges of HFS. Finally, a target current (e.g., within a range of about 10 mA to about 80 mA) can be specified such that the target current is sufficiently high for the patient to receive a therapeutic benefit (e.g., TOMAC) while also sufficiently low as to not cause discomfort or other side effects.

The above specified parameters—driving HFS driving HFS, at high total energy, with rapid up/down slew-rates, into a physiological load with an inherently capacitive property, can result in uniquely high amounts of AC Reactive Load in a medical device as compared to other TENS or implantable devices. For example, a combined (e.g., resistive and reactive) nature of the target tissue can reflect or return power to the therapy-producing circuit, which must dissipate the resulting heat; yet also stay below desired temperature thresholds to promote a safety of a body-worn medical device.

In an example, the neurostimulation device 202 can include a thermal management system configured to address the above-identified thermal challenges with producing the TOMAC excitation waveform. For example, the electrostimulation electronics unit 204 can include or use a heat sink or insulating cover arranged such as to receive thermal conduction from components (e.g., transistors, switches, etc.) of the PCB 228. For example, the heat sink can include a dispersion structure integrated into the flex circuit 222 that also includes the interconnect 232 for the electrode terminals 208. Examples of such a heat sink and thermal management system are included in US Provisional Application Ser. No. 63/585,761 filed on Aug. 24, 2024 and entitled “WEARABLE DEVICE FOR PRODUCING HIGH FREQUENCY ELECTROSTIMULATION”, which is incorporated by reference herein for its teaching of thermal management of an electrostimulation electronics unit.

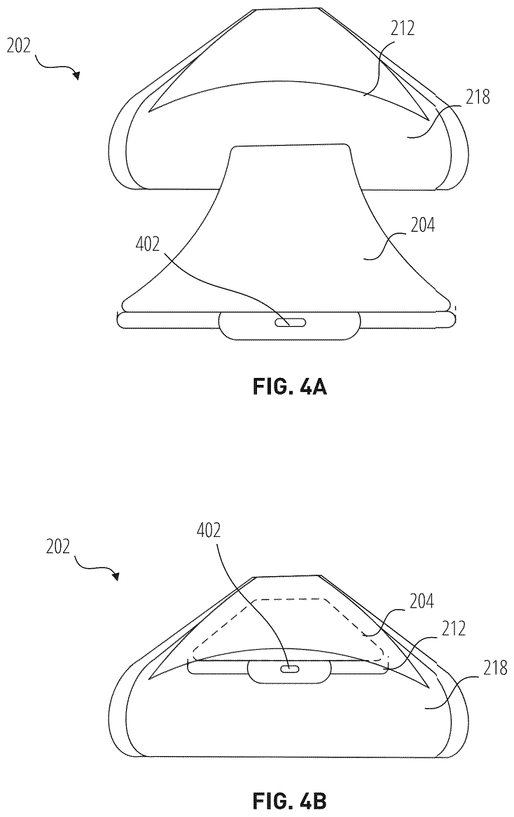

FIG. 4A depicts an electrostimulation electronics unit from an end user point of view for insertion into an opening of a removable strap. FIG. 4B depicts an assembled electrostimulation device, including an electrostimulation electronics unit having been user-inserted into a removable strap, from an end user point of view. As described above with respect to FIG. 2A, FIG. 2B, and FIG. 2C, the electrostimulation electronics unit 204 can be insertable by an end user into an opening 212 of a pocket, defining an elongate cavity, of the removable strap 218. Forming an elongate housing of an electrostimulation electronics unit 204 such that an end user can easily and repeatedly insert the elongate housing into the elongate cavity, while maintaining adequate flexibility of the elongate housing for use in wearable neurostimulation around a patient limb, can be challenging. For example, the elongate housing of the electrostimulation electronics unit 204 can essentially act as an “introducer” for being pushed from one end to position, namely, electrodes or electrode terminals included on a surface of the housing with respect to corresponding apertures in the removable strap 218. In an example, the elongate housing can be flexible, yet sized, shaped, and formed of a material such that it is sufficiently rigid for inserting into the opening 212 and elongate cavity of the removable strap 218 without buckling or bending in two or more directions during the insertion. In an example, the electrostimulation electronics unit 204 can include any user-accessible electronic connections (e.g., a port 402) at a proximal end (as depicted in FIG. 4A and FIG. 4B), such that such an electronic connection can still be user-accessible following insertion of the electrostimulation electronics unit 204 into the removable strap 218.

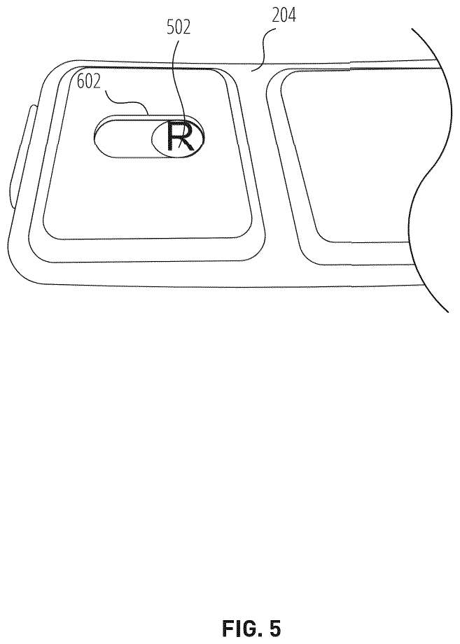

FIG. 5 depicts an example of a display of an electrostimulation device. In an example, the electrostimulation electronics unit can provide a viewing window to a display 502. The electrostimulation electronics unit 204 can display, e.g., a specified limb setting of the electrostimulation electronics unit 204 to an end user. The specified limb setting can include a type of limb, a chirality of limb (e.g., left or right) or an intended placement location on a limb (e.g., upper leg, lower leg, etc.). In an example, the wearable strap can include a display aperture, positioned to reveal the display 502 of the electrostimulation electronics unit. Here, the display 502 can be visible to the end user even when the band is pulled, tightened, bent, or otherwise distorted in shape, e.g., maintaining the viewing window based on the deflection force from an interface between an affixation feature of the wearable strap and a corresponding protrusion of the electrostimulation electronics unit.

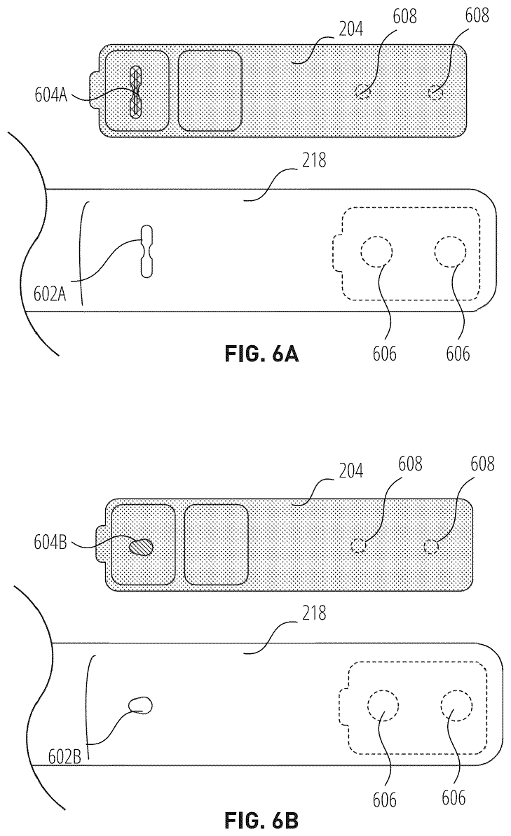

FIG. 6A and FIG. 6B each depict an example of an affixation feature of a removable strap, sized and shaped to pair with a corresponding aperture of an electrostimulation electronics unit. In an example, the removable strap 218 can include an affixation feature 602, sized and shaped such as to pair with a corresponding feature 604 (e.g., a protrusion) on the electrostimulation electronics unit 204. The affixation feature 602 can help facilitate precise alignment and secure attachment of the electrostimulation electronics unit within the cavity of the removable strap 218. Such an affixation feature 602, when paired with the corresponding feature 604 of the electrostimulation electronics unit 204, can exhibit physical or audible feedback during pairing, such as a tactile or audible click or pop perceived by the end user when features 602 and 604 are engaged with each other. The relative shape and size of the electrostimulation electronics unit 204, the cavity within the removable strap 218, and the features 602 and 604 can facilitate and maintain a specified device position, such as when the removable strap 218 is subjected to shear forces within a range of about 0.4 to about 45 newtons or a bending deflection within a range of about 3.5-inch diameter to about 9-inch diameter.

As shown in FIG. 6A, the affixation feature 602A of the wearable strap 218 and a protrusion 604A of the electrostimulation electronics unit 204 can each be oblong in shape. The wearable strap 218 can also include one or more electrode apertures 606 (e.g., arranged on an opposite side of the strap as the affixation feature 602A), arranged on the wearable strap 218 at a distance from the affixation feature 602A, such that when the protrusion 604A of the electrostimulation electronics unit 204 reaches the affixation feature 602A of the wearable strap 218, corresponding electrodes 608 (e.g., electrode terminals) included on an elongate housing of the unit 204 become aligned with respective electrode apertures 606 (e.g., at the opposite side of the wearable strap 218). The interface between the protrusion 604A and the affixation feature 602A can create an interference that deflects under force, such as to maintain a relative position of the electrostimulation electronics unit 204 within the wearable strap 218, even when subject to bending, pulling, and other transformations of the wearable strap. In an example, as depicted in FIG. 6B, an affixation feature 602B of the wearable strap 218, a protrusion 604B of the electrostimulation electronics unit 204, or both can be asymmetrically shaped, such as formed in a shape not having rotational symmetry (i.e., with respect to rotation on a plane defined by the wearable strap 218 or the electrostimulation electronics unit 204) (as contrasted with the features 602A and 602B of FIG. 6A, which are formed in a shape having rotational symmetry). Such a biased, oblong shape of features 602B and 604B can facilitate an audible or tactile click or pop when the features 602B and 604B become engaged with each other.

What is claimed is:

1. A wearable, replaceable garment for holding a battery-powered neurostimulation device at an external target location on patient skin, the wearable garment comprising:
a thermal insulation layer, including a textile, configured be disposed between and reduce thermal transfer from a portion of the neurostimulation device and to the patient skin;
a pocket defining a cavity sized and shaped to at least partially encase the neurostimulation device therewithin;
an electrode aperture, defined by the pocked, the electrode aperture arranged to expose one or more electrodes of the neurostimulation device to the external target location; and
an affixation feature configured to pair with a corresponding feature of the neurostimulation device, the affixation feature configured align and attach the neurostimulation device at a specified location within the cavity;
wherein the cavity and the affixation feature are configured to maintain attachment and alignment of the neurostimulation device at the specified location when subject to at least one of a specified shear force or specified bending deflection on the wearable garment.

2. The wearable garment of claim 1, wherein the affixation feature includes an affixation aperture, sized and shaped to configured to receive an anchor projection of the neurostimulation device.

3. The wearable garment of claim 2, wherein the electrode aperture is arranged to maintain a relative position to that of the affixation feature when the garment is under substantial bending deflection.

4. The wearable garment of claim 2, including a viewing window for revealing a display on the anchor projection of the neurostimulation device, the display configured for a specified limb setting of the neurostimulation device to an end user.

5. The wearable garment of claim 4, wherein the alignment of viewing window is medially arranged on the garment and the affixation feature, the viewing window, and the electrode aperture are configured to stabilize the neurostimulation device within the garment to reveal the display indication when the garment is under substantial bending deflection.

6. The wearable garment of claim 1, wherein the thermal insulation layer includes a foam including at least one of a polyethylene foam, a polyurethane foam, a polystyrene foam, or an ethylene-vinyl acetate foam.

7. The wearable garment of claim 1, wherein the specified shear force is within a range of 0.4 newtons (N) and 45 N.

8. The wearable garment of claim 1, wherein the specified bending deflection is a planar deflection within a range of 3.5-inch diameter and 9-inch diameter.

9. A neurostimulation device for treating at least one of RLS or PLMD, the device comprising:
a flexible, elongate housing, sized and shaped to be user-inserted into a cavity of an elongate strap at a specified internal position with respect to at least two apertures of the elongate strap, the elongate housing including:
a printed circuit board (PCB) including a neurostimulation electrical waveform generator, configured to generate a tonic motor activation (TOMAC) excitation waveform to elicit TOMAC in a patient limb during delivery of the waveform by electrodes to patient skin, the waveform configured for delivery at a constant current, a frequency in a range of 350 Hz to 10,000 Hz, a zero-to-peak current amplitude within a range of 10 milliamperes (mA) and 80 mA; and
an electronics battery;
wherein the PCB and the electronics battery are arranged at different locations along a length of the elongate housing to allow articulation between electronics housing and the battery housing.

10. The neurostimulation device of claim 9, wherein the flexible elongate housing is formed of a material having a rigidity for insertion of the elongate housing, by an end-user, into the cavity to position therapy output electrodes and to resist buckling during the insertion.

11. The neurostimulation device of claim 9, wherein the elongate housing at least partially houses the electrodes and is configured for delivery of the waveform, without needing concurrent electrical connection with any external device outside of the elongate housing, through at least one electrode aperture of the elongate strap and to the patient skin.

12. A limb wearable neurostimulation system for treating at least one of RLS or PLMD, the system comprising:
an elongate strap, of a length to substantially encircle the limb, the strap configured to hold electrodes electrically connected to an electrostimulation electronics unit against patient skin, the elongate strap including:
a pocket defining a cavity; and
an affixation feature configured to pair with a corresponding feature of the electrostimulation electronics unit, the affixation feature configured align and attach the unit at a specified location within the cavity;
the electrostimulation electronics unit, including a flexible, elongate housing, sized and shaped to be inserted into the cavity, the elongate housing including the electrodes and enclosing:
a printed circuit board (PCB) including a neurostimulation electrical waveform generator, configured to generate a tonic motor activation (TOMAC) excitation waveform to elicit TOMAC in the limb during delivery of the waveform by electrodes to patient skin, the waveform configured for delivery at a constant current, a frequency in a range of 350 Hz to 10,000 Hz, a zero-to-peak current amplitude within a range of 10 milliamperes (mA) and 80 mA; and
a battery;
wherein the PCB and the electronics battery are internally arranged at different locations along a length of the elongate housing to allow articulation between electronics housing and the battery housing.

13. The system of claim 12, wherein the flexible elongate housing is formed of a material having a rigidity for insertion of the elongate housing, by an end-user, into the cavity to position therapy output electrodes and to resist buckling during the insertion.

14. The system of claim 13, wherein the affixation feature includes an affixation aperture, sized and shaped to configured to receive an anchor projection of the electrostimulation electronics unit.

15. The system of claim 14, wherein the elongate strap includes an electrode aperture, arranged to maintain a relative position to that of the affixation feature when the elongate strap is under a specified bending deflection.

16. The system of claim 15, wherein the elongate strap includes a viewing window for revealing a display on the anchor projection of the electrostimulation electronics unit, the display configured for presenting a specified limb setting of the electrostimulation electronics unit to an end user.

17. The system of claim 16, wherein the alignment of viewing window is medially arranged on the garment and the affixation feature, the viewing window, and the electrode aperture are configured to stabilize the neurostimulation device within the garment to reveal the display indication when the garment is under substantial bending deflection.

18. The system of claim 17, wherein the elongate strap includes a thermal insulation layer, formed of foam including at least one of a polyethylene foam, a polyurethane foam, a polystyrene foam, or an ethylene-vinyl acetate foam.
